(19) United States
(12) Patent Application Publication
(10) Pub. No.: US 2025/0152050 A1
(43) Pub. Date: May 15, 2025

(54) MULTI-RATE ANALYTE SENSOR DATA COLLECTION WITH SAMPLE RATE CONFIGURABLE SIGNAL PROCESSING

(71) Applicant: ABBOTT DIABETES CARE INC., Alameda, CA (US)

(72) Inventor: Erwin Satrya Budiman, Fremont, CA (US)

(73) Assignee: ABBOTT DIABETES CARE INC., Alameda, CA (US)

(21) Appl. No.: 18/980,752

(22) Filed: Dec. 13, 2024

Related U.S. Application Data
(63) Continuation of application No. 17/098,286, filed on Nov. 13, 2020, now Pat. No. 12,171,548.
Application No. 17/098,286 is a continuation of application No. 15/292,107, filed on Oct. 12, 2016, now Pat. No. 10,874,336.
Application No. 15/292,107 is a continuation of application No. 14/210,330, filed on Mar. 13, 2014, now Pat. No. 9,474,475.
(60) Provisional application No. 61/800,878, filed on Mar. 15, 2013.

(51) Int. Cl. A61B 5/145; A61B 5/00; G16H 10/60

(52) U.S. Cl. CPC A61B 5/14532; A61B 5/72; A61B 5/7225; A61B 5/725; A61B 5/742; A61B 2562/0295; G16H 10/60

(57) ABSTRACT

Systems, methods and apparatus are provided, including one or more processors configured to receive signals generated by an analyte sensor, a memory storing instructions which, when executed by the one or more processors, cause the one or more processors to, store a first dataset corresponding to the signals generated by the analyte sensor at a first rate in a first memory buffer, and store a second dataset corresponding to the signals generated by the analyte sensor at a second rate in a second memory buffer, wherein the second rate is a dynamic value based on a rate-of-change corresponding to the signals generated by the analyte sensor.

### RELATED APPLICATIONS

The present application is a continuation under 35 U.S.C. § 120 of U.S. patent application Ser. No. 17/098,286 filed Nov. 13, 2020, which is a continuation of U.S. patent Ser. No. 15/292,107 filed Oct. 12, 2016, now U.S. Pat. No. 10,874,336, which is a continuation of U.S. patent Ser. No. 14/210,330 filed Mar. 13, 2014, now U.S. Pat. No. 9,474,475, which claims priority under 35 U.S.C. § 119(e) to U.S. Provisional Application No. 61/800,878 filed Mar. 15, 2013, entitled “Multi-Rate Analyte Sensor Data Collection with Sample Rate Configurable Signal Processing,” the disclosures of all of which are incorporated herein by reference for all purposes.

The present application is related to co-pending U.S. application Ser. No. 14/210,312 filed Mar. 13, 2014, entitled “Noise Rejection Methods and Apparatus For Sparsely Sampled Analyte Sensor Data,” U.S. application Ser. No. 14/210,303 filed Mar. 13, 2014, entitled “Analyte Sensor Data Parameterized Filtering Methods and Apparatus,” U.S. application Ser. No. 13/984,815 filed on Nov. 25, 2013, entitled “Software Applications Residing on Handheld Analyte Determining Devices” and U.S. application Ser. No. 12/807,278 filed Aug. 31, 2010, entitled “Medical Devices and Methods,” all of which are incorporated herein by reference for all purposes.

### BACKGROUND

The detection of the concentration level of glucose or other analytes in certain individuals may be vitally important to their health. For example, the monitoring of glucose levels is particularly important to individuals with diabetes or pre-diabetes. People with diabetes may need to monitor their glucose levels to determine when medication (e.g., insulin) is needed to reduce their glucose levels or when additional glucose is needed.

Devices have been developed for automated in vivo monitoring of analyte time series characteristics, such as glucose levels, in bodily fluids such as in the blood stream or in interstitial fluid. Some of these analyte level measuring devices are configured so that at least a portion of a sensor of an on-body device is positioned below a skin surface of a user, e.g., in a blood vessel or in the subcutaneous tissue of a user. As used herein, the term analyte monitoring system is used to refer to any type of in vivo monitoring system that uses a sensor disposed with at least a subcutaneous portion to measure and store sensor data representative of analyte concentration levels automatically over time. Analyte monitoring systems include both (1) systems such as continuous glucose monitors (CGMs) which transmit sensor data continuously or at regular time intervals (e.g., once per minute) to a processor/display unit and (2) systems that transfer stored sensor data in one or more batches in response to a scan request from a processor/display unit (e.g., based on an activation action and/or proximity using, for example, a near field communications protocol).

Monitoring a user's analyte level that has a relatively high rate of change can require collecting and storing a relatively large amount of sensor data. However, in an effort to minimize the cost, size, and power requirements of analyte monitoring systems, it is desirable to minimize the amount of memory required for use within the on-body device. Thus, what is needed are systems, methods and apparatus that can provide a sufficient amount of sensor data to accurately determine a user's analyte level but at the same time minimize the memory requirements.

### SUMMARY

The present disclosure provides systems, methods, and apparatus for determining an estimate of an analyte level over time. The invention includes sampling sensor data using an analyte sensor positioned at least partially subcutaneously, storing a first dataset of sensor data at a first rate, storing a second dataset of sensor data at a second rate, determining an estimated analyte level based on the first and second datasets, and outputting the estimated analyte level to a display. Storing the first and second datasets can include storing the first and second datasets concurrently and the first rate can be higher than the second rate. Storing the first dataset can also include storing the first dataset in a first memory buffer and storing the second dataset can include storing the second dataset in a second memory buffer. The first memory buffer can have a size that is different than a size of the second memory buffer. Storing the first and second datasets can include storing the first and second datasets in separate memory buffers within sensor electronics disposed with an on body device. The present disclosure further includes transferring the first and second datasets from the memory buffers to a receiver for signal processing. Storing the first and second datasets can include storing the first and second datasets in separate memory partitions of a single memory device. Determining an estimated analyte level can include determining a discrete time approximation of a filter model represented by a state space realization.

The invention also includes a computer system and a computer program product for filtering analyte monitoring system sensor data. Numerous other aspects and embodiments are provided. Other features and aspects of the present disclosure will become more fully apparent from the following detailed description, the appended claims, and the accompanying drawings.

### BRIEF DESCRIPTION OF THE DRAWINGS

The accompanying drawings, which are incorporated herein, form part of the specification. Together with this written description, the drawings further serve to explain the principles of, and to enable a person skilled in the relevant arts, to make and use the present disclosure.

FIG. 1 depicts a data monitoring and management system such as, for example, an analyte (e.g., glucose) monitoring system in accordance with some embodiments of the present disclosure.

FIG. 2 is a block diagram of a receiver/monitor unit such as that shown in FIG. 1 in accordance with some embodiments of the present disclosure.

FIG. 3 depicts an example graph illustrating a plot of analyte level over time in accordance with some embodiments of the present disclosure.

FIG. 4 depicts an example graph illustrating a plot of sensor data points collected over time in accordance with some embodiments of the present disclosure.

FIG. 5 depicts an example graph illustrating a magnified view of the analyte level plot of FIG. 3 overlaid on the sensor data point plot of FIG. 4 in accordance with some embodiments of the present disclosure.

FIG. 6 is a flow chart depicting an example method in accordance with some embodiments of the present disclosure.

### DETAILED DESCRIPTION

Before the embodiments of the present disclosure are described, it is to be understood that this invention is not limited to the particular embodiments described, as such may, of course, vary. It is also to be understood that the terminology used herein is for the purpose of describing particular embodiments only, and is not intended to be limiting, since the scope of the embodiments of the invention will be limited only by the appended claims.

The present disclosure provides systems, methods, and apparatus to monitor an analyte level using a minimized amount of memory and both a maximized time window of available past sensor data and a maximized density of recent sensor data. At the same time, the present disclosure facilitates artifact rejection and lag correction. The invention uses a combination of a hardware configuration (e.g., memory arrangement) and a signal processing method to achieve these features and benefits.

The invention can be applied to sensor data from an analyte monitoring system, such as, for example, any type of in vivo monitoring system that uses a sensor disposed with at least a subcutaneous portion to measure and store sensor data representative of analyte concentration levels automatically over time. Analyte monitoring systems can include CGMs which are programmed to transmit sensor data according to a predetermined transmission schedule, continuously, or at regular time intervals to a processor/display unit and systems that transfer stored sensor data in one or more batches in response to a request from a processor/display unit, i.e., not according to a predetermined transmission schedule.

According to some embodiments of the present disclosure, datasets related to a patient's monitored analyte concentration level (herein referred to as “sensor data”) over time are received from an on-body device that includes an analyte sensor. The sensor data can include datasets with differing data rates. In some embodiments, the sensor data can represent a collection of data that is transmitted from an on-body device at several different times during a wear period of the on-body device. In some other embodiments, the sensor data can represent data collected and stored over an entire wear period of an on-body device and only transmitted from the on-body device at the end of the wear period or at the end of the useful life of the on-body device. In other words, the sensor data can be transmitted continuously, on a regular schedule, in multiple batches over time, in batches on demand, or in a single batch.

The embodiments of the present disclosure can also be applied to any analyte concentration level determination system that may exhibit or at least be suspected of exhibiting, or that may be susceptible to noise or artifacts in the sensor data. Embodiments of the invention are described primarily with respect to continuous glucose monitoring devices and systems but the present disclosure can be applied to other analytes and analyte characteristics, as well as data from measurement systems that transmit sensor data from a sensor unit to another unit such as a processing or display unit in response to a request from the other unit. For example, other analytes that can be monitored include, but are not limited to, acetyl choline, amylase, bilirubin, cholesterol, chorionic gonadotropin, creatine kinase (e.g., CK-MB), creatine, DNA, fructosamine, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, can also be monitored. In the embodiments that monitor more than one analyte, the analytes can be monitored at the same or different times. The present disclosure also provides numerous additional embodiments.

Embodiments of the present disclosure may include a programmed computer system adapted to receive and store data from an analyte monitoring system. The computer system can include one or more processors for executing instructions or programs that implement the methods described herein. The computer system can include memory and persistent storage devices to store and manipulate the instructions and sensor data received from the analyte monitoring system. The computer system can also include communications facilities (e.g., wireless and/or wired) to enable transfer of the sensor data from the analyte monitoring system to the computer. The computer system can include a display and/or output devices for presenting the sensor data to a user. The computer system can include input devices and various other components (e.g., power supply, operating system, clock, etc.) that are typically found in a conventional computer system. In some embodiments, the computer system can be integral to the analyte monitoring system. For example, the computer system can be embodied as a handheld or portable receiver unit within the analyte monitoring system or, in some embodiments, as a processor or logic circuit within the sensor electronics of an on-body device of the analyte monitoring system.

The various methods described herein for performing one or more processes also described herein can be embodied as computer programs (e.g., computer executable instructions and data structures) developed using an object oriented programming language that allows the modeling of complex systems with modular objects to create abstractions that are representative of real world, physical objects and their interrelationships. However, any practicable programming language and/or techniques can be used. The software for performing the inventive processes, which can be stored in a memory or storage device of the computer system described herein, can be developed by a person of ordinary skill in the art based upon the present disclosure and can include one or more computer program products. The computer program products can be stored on a computer readable medium such as a server memory, a computer network, the Internet, and/or a computer storage device. Note that in some cases the methods embodied as software may be described herein with respect to a particular order of operation or execution. However, it will be understood by one of ordinary skill that any practicable order of operation or execution is possible and such variations are contemplated by this specification of the present disclosure.

Filtering noise, compensating for undesired dynamics, and responding to variable availability of data points can be significant in generating an accurate representation of an analyte concentration level using an analyte monitoring system. In some analyte monitoring systems, for example, the sensor data can include a window of sampled data long enough to cover a significant portion of a day, e.g., a 6 to 24 hour window with data points collected every 10 to 20 minutes. However, significant changes in the measurement of an analyte level can occur in less than 10 minutes which such systems may not be able to detect. In addition to filtering noise and artifacts, some of the data points may not be available due to data quality issues. A reliable analyte measurement system according to the present disclosure can filter noise, correct for artifacts, compensate for undesired dynamics, and recover missing data using the remaining data.

In an analyte measurement system adapted to determine and represent a user's glucose concentration level, for example, a filter is used to process sensor data received as signals by the sensor electronics within the on-body device. A component of the filter utilizes several pre-conditioned sensor signals, z, at various time instances, to generate an output that represents the best estimate of reference glucose and reference glucose rate of change values.

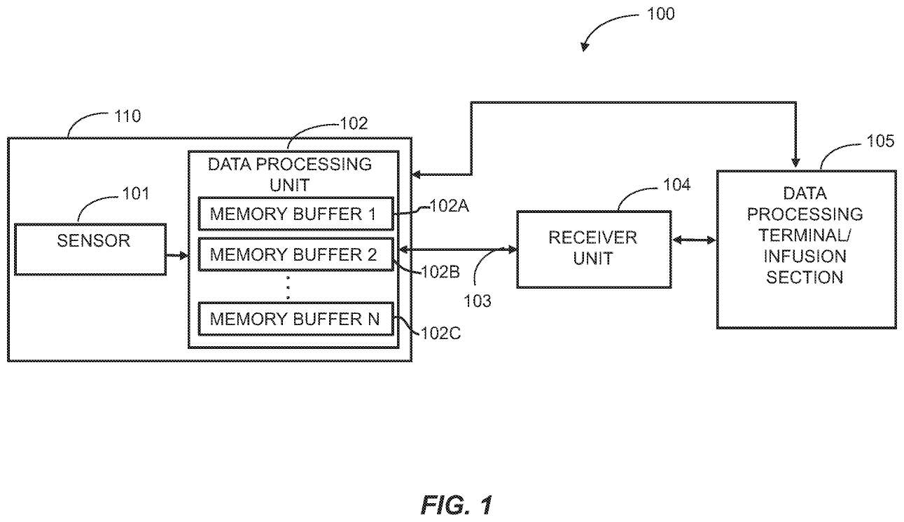

FIG. 1 depicts an illustrative embodiment of a data monitoring and management system such as, for example, an analyte (e.g., glucose) monitoring system in accordance with the present disclosure. Note that the present disclosure is frequently described herein with reference to diabetes treatment based on measurement and control of glucose levels using insulin, however, the present disclosure is applicable to treatment of many different diseases based on measurement and/or control of many different analytes using many different medications. As shown in FIG. 1, an analyte monitoring system 100 can include a sensor 101, a data processing unit (e.g., sensor electronics) 102 connectable to the sensor 101, and a receiver unit 104 which is configured to communicate with the data processing unit 102 via a communication link 103. In some embodiments of the present disclosure, the sensor 101 and the data processing unit (e.g., sensor electronics) 102 can be configured as a single integrated assembly 110. In some embodiments, the integrated sensor and sensor electronics assembly 110 can be configured as an on-body device wearable by a user. In such embodiments, the on-body device can be configured, for example, for wireless radio frequency identification (RFID) and/or radio frequency (RF) communication with a reader device/receiver unit 104, and/or an insulin pump.

In some embodiments, the receiver unit 104 can be further configured to transmit data to a data processing terminal 105 to evaluate or otherwise process or format data received by the receiver unit 104. The data processing terminal 105 can be configured to receive data directly from the data processing unit 102 via a communication link which can optionally be configured for bi-directional communication. Further, the data processing unit 102 can include a transmitter or a transceiver to transmit and/or receive data to and/or from the receiver unit 104, the data processing terminal 105.

Only one sensor 101, data processing unit 102, and data processing terminal 105 are shown in the embodiment of the analyte monitoring system 100 illustrated in FIG. 1. However, it will be appreciated by one of ordinary skill in the art that the analyte monitoring system 100 can include more than one sensor 101 and/or more than one data processing unit 102, and/or more than one data processing terminal 105.

The analyte monitoring system 100 can be a continuous monitoring system, or semi-continuous, or a discrete monitoring system. In a multi-component environment, each component can be configured to be uniquely identified by one or more of the other components in the system so that communication conflict can be readily resolved between the various components within the analyte monitoring system 100. For example, unique IDs, communication channels, and the like, can be used.

In certain embodiments, the sensor 101 is physically positioned in or on the body of a user whose analyte level is being monitored. The data processing unit 102 is coupleable to the sensor 101 so that both devices are positioned in or on the user's body, with at least a portion of the analyte sensor 101 positioned transcutaneously. The data processing unit 102 in certain embodiments can include a portion of the sensor 101 (proximal section of the sensor in electrical communication with the data processing unit 102) which is encapsulated within or on a printed circuit board of the data processing unit 102 with, for example, potting material or other protective material. The data processing unit 102 performs data processing functions, where such functions can include but are not limited to, storing, filtering and encoding of data signals, each of which corresponds to a sampled analyte level of the user, for transmission to the receiver unit 104 via the communication link 103. In some embodiments, the sensor 101 or the data processing unit 102 or a combined sensor/data processing unit can be wholly implantable under the skin layer of the user.

In some embodiments, the receiver unit 104 can include an analog interface section including an RF receiver and an antenna that is configured to communicate with the data processing unit 102 via the communication link 103, and a data processing section for processing the received data from the data processing unit 102 such as data decoding, error detection and correction, data clock generation, and/or data bit recovery.

Referring still to FIG. 1, the data processing terminal 105 can include an infusion device such as an insulin infusion pump or the like, which can be configured to administer insulin to patients, and which can be configured to communicate with the receiver unit 104 for receiving, among others, the measured analyte level. Alternatively, the receiver unit 104 can be configured to integrate an infusion device therein so that the receiver unit 104 is configured to administer insulin (or other appropriate drug) therapy to patients, for example, for administering and modifying basal profiles, as well as for determining appropriate boluses for administration based on, among others, the detected analyte levels received from the data processing unit 102. An infusion device can be an external device or an internal device (wholly implantable in a user). An insulin bolus calculator can be operatively coupled to the receiver unit 104 to determine an insulin dose that is required based upon the analyte data received from the sensor device/electronics.

In some embodiments, the data processing terminal 105, which can include an insulin pump, can be configured to receive the analyte signals from the data processing unit 102, and thus, incorporate the functions of the receiver unit 104 including data processing for managing the patient's insulin therapy and analyte monitoring.

In some embodiments, the data processing unit 102 (e.g., the sensor electronics) can include one or more memory buffers 102A, 102B, 102C used by the data processing unit 102 to store sensor data from the sensor 101. The memory buffers 102A, 102B, 102C can be embodied as first-in-first-out (FIFO) buffers that can hold one or more sets of recent sensor data so that the sensor data can be transferred to the receiver unit 104. The sets of sensor data can each represent a moving time window of data points collected or sampled by the sensor. As time passes, new data is stored in the memory buffers 102A, 102B, 102C and older data is overwritten such that at any given time, a memory buffer 102A, 102B, 102C holds a collection of the most recent data points received and extending back in time based on the size of the buffer and the storage rate.

The storage rate at which the sensor data is written into the memory buffers 102A, 102B, 102C can correspond to the sampling rate of the sensor or a selected lower rate. The size of the time window of data points can correspond to the size of the associated memory buffer 102A, 102B, 102C selected to store the data at the storage rate. In some embodiments, different memory buffers 102A, 102B, 102C can be different sizes and can store sensor data at different rates. In other words, the density of the data (e.g., the number of data points stored in a given amount of time) in a particular memory buffer can be different for different memory buffers 102A, 102B, 102C.

In some embodiments, the sampling rate of the sensor is constant and the storage rates in the memory buffers are based on a selected fraction of the sampling rate. For example, where the sensor sampling rate is once every minute; the storage rate in a first memory buffer 102A can also be once every minute while the storage rate in a second memory buffer 102B can be once every twenty minutes. If both buffers 102A, 102B each include, for example, 0.5 Kb (i.e., 512) of storage addresses, the first memory buffer 102A can hold an approximately 8.5 hour time window of data points while the second memory buffer 102B can hold an approximately seven day time window of data points. In some embodiments, the sampling rate may be once every two minutes, or once every five minutes, or other suitable sampling rate, and in which case, the storage rate in the first memory buffer 102 and the storage rate in the second memory buffer 102B may correspondingly change to different suitable rates.

In some embodiments, the storage rate can be varied. For example, the rate at which the data processing unit 102 stores data into one or more of the memory buffers 102A, 102B, 102C can be set based upon a selected variable. In some embodiments, based upon the rate of change of values of the sensor data, it may be desirable to store more or less data per unit time. For example, where the value of the sensor data is relatively constant, the sensor data may only be stored infrequently and where the sensor data is changing rapidly, the sensor data may be stored relatively frequently. In such embodiments, information that can be used to determine the storage rate (e.g., timestamp, time between data points, etc.) can also be stored. This allows the amount of memory to be minimized while still providing increased sensor data density when significant events may be occurring.

In some embodiments, the memory buffers 102A, 102B, 102C can be implemented as different memory devices (e.g., different memory integrated circuits) or as a singled memory device partitioned into different sections. Any practicable type of memory can be used to implement the memory buffers 102A, 102B, 102C.

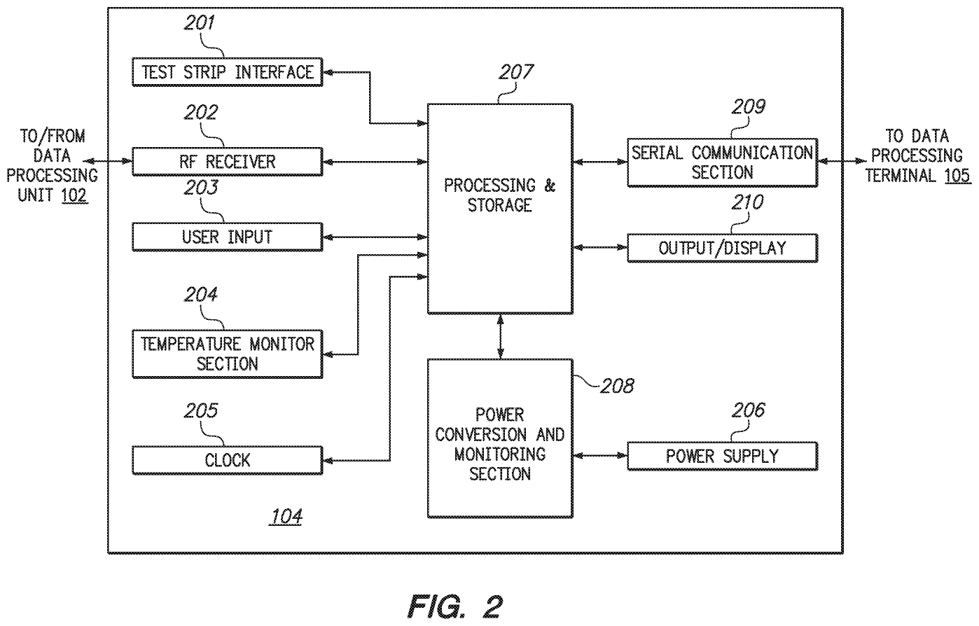

FIG. 2 is a block diagram of a receiver/monitor unit 104 or insulin pump such as that shown in FIG. 1 in accordance with certain embodiments. The receiver unit 104 can include one or more of: a blood glucose test strip interface 201, an RF receiver 202, an input 203, a temperature detection section 204, and a clock 205, each of which is operatively coupled to a processing and storage section 207. In certain embodiments, a receiver, such as receiver unit 104, also includes a power supply 206 operatively coupled to a power conversion and monitoring section 208. Further, the power conversion and monitoring section 208 is also coupled to the receiver's processor 207. Moreover, also shown are a receiver serial communication section 209, and an output 210, each operatively coupled to the processing and storage unit 207. The receiver unit 104 can include user input and/or interface components or can be free of user input and/or interface components.

In some embodiments, the RF receiver 202 is configured to communicate, via the communication link 103 (FIG. 1) with the data processing unit (e.g., sensor electronics) 102, to receive encoded data from the data processing unit 102 for, among others, signal mixing, demodulation, and other data processing. The input 203 of the receiver unit 104 is configured to allow the user to enter information into the receiver unit 104 as needed. In one aspect, the input 203 can include keys of a keypad, a touch-sensitive screen, and/or a voice-activated input command unit, and the like. The temperature monitor section 204 can be configured to provide temperature information of the receiver unit 104 to the processing and control section 207, while the clock 205 provides, among others, real time or clock information to the processing and storage section 207.

Each of the various components of the receiver unit 104 shown in FIG. 2 is powered by the power supply 206 (or other power supply) which, in some embodiments, includes a battery. The output/display 210 of the receiver unit 104 is configured to provide, among others, a graphical user interface (GUI), and can include a liquid crystal display (LCD) for displaying information and/or allowing a user to enter information. The receiver unit 104 can also include a storage section such as a programmable, non-volatile memory device as part of the processor 207, or provided separately in the receiver unit 104, operatively coupled to the processor 207.

Conventional analyte monitors typically use a single memory buffer to store sensor data at a single rate. To be able to accurately represent an analyte level over time however, including correcting for lag, filtering noise and artifacts, and correcting for missing or unavailable data points, such a system would need to store data at a relatively fast rate (e.g., once per minute). Further, in order to attain a longer historical duration of analyte values, a conventional on body unit would need to be able to store a relatively large amount of data. Together, these requirements result in an on body device that includes an undesirably large memory capacity. For example, if the required data density requires a sample rate of once per three minutes, and twenty-four hours of duration needs to be covered, then the memory buffer must store 1+(24*(60/3))=481 data points. Then, conventional signal processing method that assumes a constant sample rate can be used.

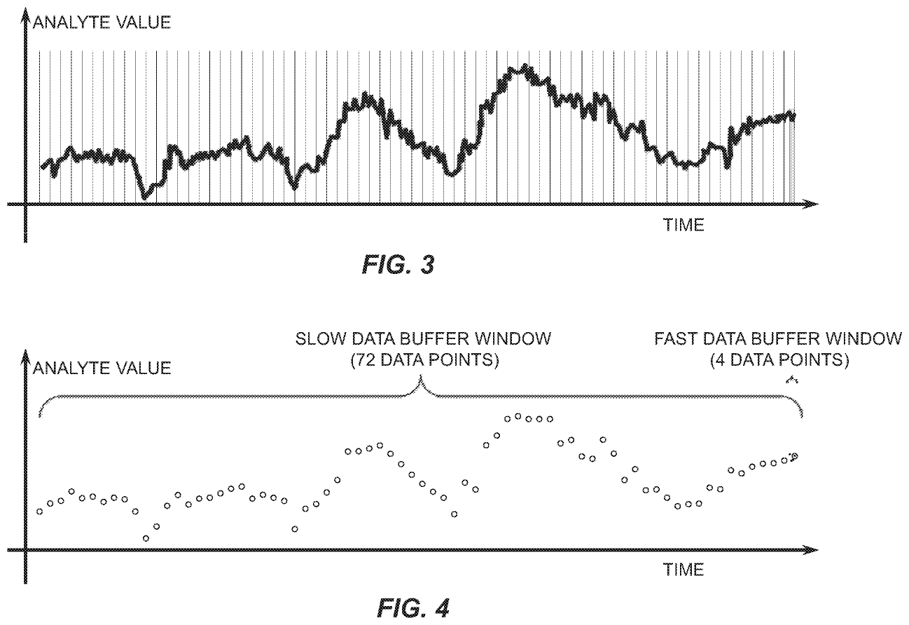

The present disclosure overcomes these requirements by providing two or more memory buffers within the on body device that are configured to store sensor data at two different rates, coupled with a signal processing method that can operate on more than one sample rate setting. In some embodiments, one relatively slow data rate over a relatively long duration is taken to maximize coverage of past data, and one relatively fast data rate over a relatively short duration is taken to maximize density of recent data. One memory buffer stores data collected at the slow rate, and another memory buffer stores data collected at the fast sample rate. For example, as illustrated in FIG. 3, consider a first memory buffer that stores sensor data at a relatively slow rate (e.g., once per 20 minutes) over a relatively long period (e.g., 24 hours) so that a large time window of data points (e.g., 72 data points) is available using a relatively small memory. A second memory buffer stores data at a relatively fast rate (e.g., once per three minutes) over a relatively short period (e.g., 9 minutes) so that detailed sensor data is available but also using only a relatively small memory (e.g., 4 data points). This arrangement allows for both long coverage of historical analyte data and high density data that is useful to generate accurate real-time analyte level output without requiring a large memory capacity within the on-body device. Compared with the conventional single rate method which would store 481 data points, the present disclosure can estimate the analyte just as accurately with only storing 1+(24*(60/20))+1+(9/3)=77 data points, i.e., an approximately 84% reduction in this example.

The present disclosure also includes providing methods of combining sensor data of different rates so that the analyte monitoring system can accurately filter the sensor data and correct for lag. For example, the calculated analyte value y is a function of the preconditioned sensor data z and calculated rates of change at different sample rates;

$$y ( k ) = z ( k ) + a 1 v 1 + a 2 v 2 + … + aN vN ,$$

with each v1 through vN calculated by

$$[ z ( k ) - z ( k - T 1 ) ] / T 1 , [ z ( k ) - z ( k - T 2 ) ] / T 2 , … , [ z ( k ) - z ( k - TN ) ] / TN$$

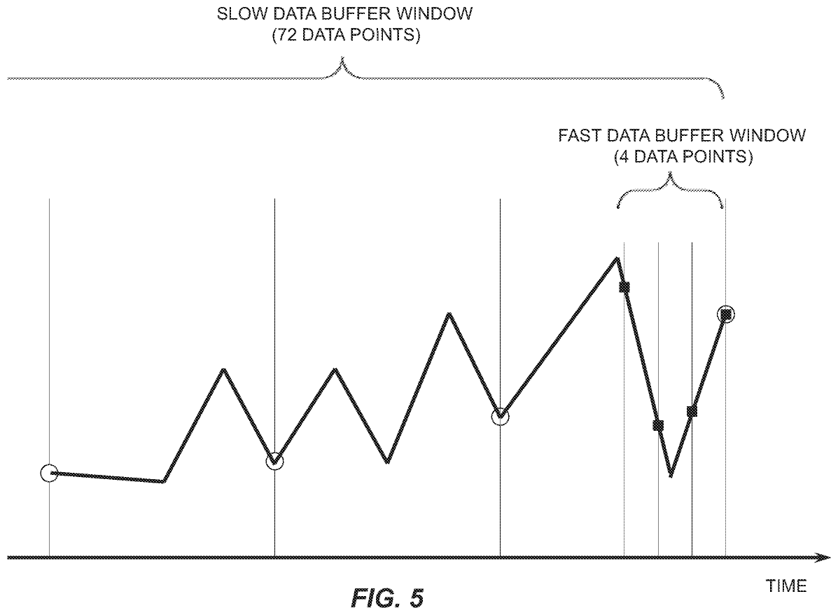

For any time instance where an output value is to be determined, a combination of nearby sensor data and a dynamic model describing the system is used to calculate the output. FIG. 4 and the magnified view provided in FIG. 5 illustrate the use of input data stored in the manner described, converted into output values at various instances.

In another example, if the latest 30 minutes are to be covered with relatively high data density, then a sample rate of once per 3 minutes will mean the first memory buffer, Fast Data buffer, to store 1+(30/3)=11 data points. For 10 hours of past data coverage with a rate of once per 20 minutes, the second memory buffer, Slow Data buffer, will store 1+(10*(60/20))=31 data points. Then, the total memory buffer size is for storing 42 data points. This is significantly less than the conventional method.

In another example, if the latest 15 minutes are to be covered with an even higher data density relative to the example above, then a sample rate of once every minute will mean the Fast Data buffer will store 1+(15/1)=16 data points. For 8 hours of past data coverage with a rate of once per 15 minutes, the Slow Data memory buffer will store 1+(8*(60/15))=33 data points. Analyte values near the sensor measured in vivo are stored into the two memory storage buffers as input data for the signal processing method. If memory storage with increments in bytes is used, reserving 2 bytes for Fast Data and 4 bytes for Slow Data yields 16 and 32 data points, respectively.

In another embodiment, a single memory buffer can store data and a time index (e.g., a timestamp, a delta time between data points, etc.), wherein the time interval from one sample to the next is determined to optimize data storage efficiency. Previously incorporated U.S. Provisional Application Ser. No. 61/485,840 describes details of how such an arrangement can be implemented.

In some embodiments, the basic calculation can be derived a priori based on a dynamic model in the continuous time domain. For example, let the output y at time t, y(t), be a function of a set of internal states x(t) and a set of sensor data u(t), described in terms of state space as:

$$\begin{matrix}y ( t ) = C \times ( t ) \\ dx ( t ) / dt = A \times ( t ) + B u ( t )\end{matrix}$$

Assuming time invariant parameter matrices A, B, and C, design of the filter in terms of noise rejection and dynamic compensation such as lag correction can be achieved using loop shaping theory or other methods, noting that the Laplace transform of the state space model above is:

$$Y ( s ) = {C [ sI - A ]}^{- 1} B U ( s )$$

Then, for the case where two memory buffers with different sample times store sensor data, the output y at discrete time instance k can be calculated by using a discrete time approximation of an infinite impulse response (IIR) filter model represented by the state space realization above. The sample time is a function of the time interval between the nearest sensor data points eligible for the output at time k.

In another example and without loss of generality, suppose x(t) and u(t) are both single dimensional time series, so that A, B, and C are scalars. Let A=−0.1, B=0.1, C=1, values which are determined a priori during the system development. One discrete time realization of this system, using zero order hold, for any discrete time instance k is then

$$y ( k ) = Ad y ( k - 1 ) + Bd u ( k - 1 ) ,$$

where:

$$Ad = {e}^{A * Ts}$$

$$and$$

$$BD = [ Ad - 1 ] * B / A .$$

Note that the discrete time parameters Ad and Bd are now dependent on the sample time Ts (=time spacing between adjacent data points). The output calculation y now depends on a model whose parameters can vary from one instance to another. If the nearby data points are all from Fast Data collected, e.g., once per minute, but the nearest available raw data is 3 minutes away, letting Ts=3 minutes, Ad=1.35, Bd=−0.35. If the nearby data points are one from Fast Data, and another from Slow Data, and the time interval happens to be 8 minutes, Ad=2.23, Bd=−1.23. Note that for models requiring more than two input data points, the state space parameters do not remain scalars.

While the previous example utilizes a state space model framework, other digital filter structures (e.g., described in terms of a Finite Impulse Response (FIR) filter) can be used. Unlike the state space derived filter, synthesis of FIR parameters given varying sample times is more complex. An example method for synthesizing parameters using the FIR approach is described in previously incorporated U.S. application Ser. No. 14/210,303 entitled “Analyte Sensor Data Parameterized Filtering Methods and Apparatus.”

In some embodiments, when a single memory buffer with irregular sample time is used, a method based on regularization can be used to calculate the output. Examples of the method of regularization are described in Numerical Recipes in C: The Art of Scientific Computing, 2 ed., Cambridge University Press, 2002, by W.H. Press, et al. and in “Nonparametric input estimation in physiological systems: Problems, methods, and case studies,” Automatica, vol. 33, pp. 851-870, 1997, by G. DeNicolao, et al., which are hereby incorporated herein by reference for all purposes. Regularization allows for each of the source data to be assigned different levels of confidence, wherein the smoothed output at any time instance is more likely to match the source data at that time instance when the source data point has a larger confidence (or equivalently, a smaller assumed measurement error model) relative to other neighboring source data points.

For robustness against artifacts, more than one estimate of the output at any instance k can be calculated using different combinations of sensor data. Alternatively, the dynamic model assumes a processed sensor data as its input stream, with this input stream made available at a desired interval, and the processed sensor data at those desired intervals are generated from sensor data with different storage rate intervals. An example method for generating processed sensor data is described in previously incorporated U.S. application Ser. No. 14/210,312 entitled “Noise Rejection Methods and Apparatus For Sparsely Sampled Analyte Sensor Data.”

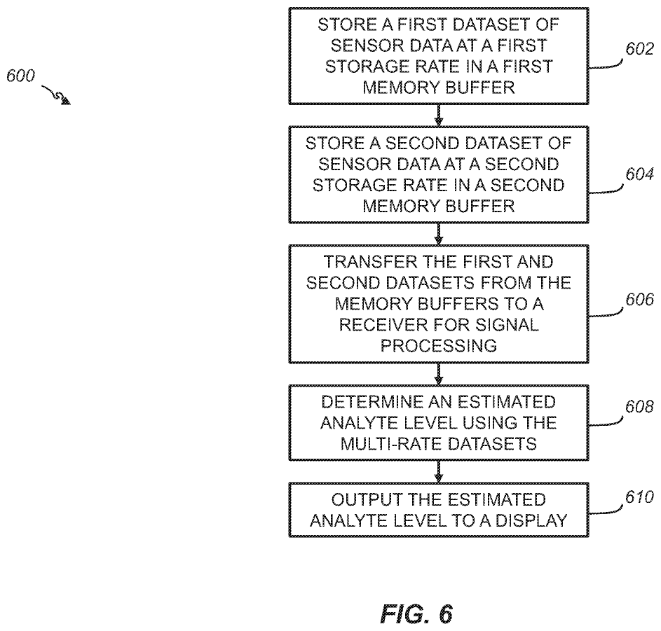

Turning now to FIG. 6, a flow chart 600 depicting example methods of the present disclosure is provided. As indicated above, the methods of the present disclosure can be implemented on a computer or other processing device. Also, the particular order in which the steps of the invention are presented here does not represent the only order in which the steps can be performed.

In some embodiments, a first dataset of sensor data is stored at a first storage rate in a first memory buffer (602) and a second dataset of sensor data is stored at a second storage rate in a second memory buffer (604). The datasets can be stored concurrently. The storage rates are different and the memory buffers are also different. For example, the first rate can be a relatively fast rate while the second rate can be a relatively slow rate. The first memory buffer can be smaller than the second memory buffer. Sensor data is stored in the memory buffers over different periods of time. For example, the sensor data stored in the first memory buffer at the relatively fast rate can be stored over a relatively short time period after which the first buffer begins to get overwritten. Meanwhile the sensor data stored in the second memory buffer at the relatively slow rate can be stored over a relatively long time period after which the second buffer begins to get overwritten. The first and second datasets are transferred from the memory buffers to a receiver for signal processing (606).

Using the multi-rate datasets, an estimated analyte level is next determined (608). A composite dataset can be generated based on a selection of data points from the first and second datasets. Filter parameters are then determined using the storage rates of the selected data points in the composite dataset. In some embodiments, a predetermined dynamic filter model in the continuous time domain is used. For example, let the output y at time t, y(t), be a function of a set of internal states x(t) and a set of sensor data u(t), described in terms of state space as:

$$\begin{matrix}y ( t ) = C \times ( t ) \\ dx ( t ) / dt = A \times ( t ) + B u ( t ) .\end{matrix}$$

Then the output y at discrete time instance k can be calculated by using a discrete time approximation of the filter model represented by the above state space realization. The storage time is a function of the time interval between the nearest sensor data points eligible for the output at time k. More generally, an estimated analyte level is determined based on one or more filters using the set of filter parameters and the composite dataset. The estimated analyte level is then output to a display (610).

In the manner described above, in certain embodiments of the present disclosure, there is provided a method comprising: sampling sensor data using an analyte sensor, storing a first dataset of the sensor data at a first rate, storing a second dataset of the sensor data at a second rate, determining an estimated analyte level based on the first and second datasets of the sensor data, and outputting the estimated analyte level to a display.

In certain embodiments, storing the first and second datasets of the sensor data includes storing the first and second datasets of the sensor data concurrently, and the first rate is higher than the second rate.

In certain embodiments, storing the first dataset of the sensor data includes storing the first dataset of the sensor data in a first memory buffer, storing the second dataset of the sensor data includes storing the second dataset of the sensor data in a second memory buffer, and the first memory buffer has a size that is different than a size of the second memory buffer.

In certain embodiments, storing the first and second datasets of the sensor data includes storing the first and second datasets of the sensor data in separate memory buffers within sensor electronics disposed with an on body device.

In certain embodiments, the method further comprises transferring the first and second datasets of the sensor data from the separate memory buffers to a receiver for processing.

In certain embodiments, storing the first and second datasets of the sensor data includes storing the first and second datasets of the sensor data in separate memory partitions of a single memory device.

In certain embodiments, determining the estimated analyte level includes determining a discrete time approximation of a filter model represented by a state space realization.

In certain embodiments, determining the estimated analyte level includes: generating a composite dataset based on a selection of all or a subset of data points from the first and second datasets of the sensor data; determining a set of filter parameters based on rates of the composite dataset, and determining the estimated analyte level based on one or more filters using the set of filter parameters and the composite dataset.

A method in certain embodiments comprises sampling sensor data using an analyte sensor, storing a plurality of datasets of the sensor data, each at a different rate, determining an estimated analyte level based on the plurality of datasets of the sensor data, and outputting the estimated analyte level to a display.

In certain embodiments, storing the plurality of datasets of the sensor data includes storing the plurality of datasets of the sensor data, each in a different memory buffer.

In certain embodiments, storing the plurality of datasets of the sensor data includes storing each dataset in separate memory buffers within sensor electronics disposed with an on body device.

In certain embodiments, the method further comprises transferring the plurality of datasets of the sensor data from the separate memory buffers to a receiver for signal processing.

In certain embodiments, storing the plurality of datasets of the sensor data includes storing the plurality of datasets of the sensor data in separate memory partitions of a single memory device.

In certain embodiments, determining the estimated analyte level includes determining a discrete time approximation of a filter model represented by a state space realization.

In certain embodiments, determining the estimated analyte level includes: generating a composite dataset based on a selection of all or a subset of data points from the plurality of datasets of sensor data, determining a set of filter parameters based on rates of the composite dataset, and determining the estimated analyte level based on one or more filters using the set of filter parameters and the composite dataset.

A system for determining an estimate of an analyte level over time in certain embodiments comprises a processor, and a memory coupled to the processor, the memory storing processor executable instructions to: sample sensor data using an analyte sensor, store a plurality of datasets of the sensor data, each at a different rate, determine an estimated analyte level based on the plurality of datasets of the sensor data, and output the estimated analyte level to a display.

In certain embodiments, the memory including processor executable instructions to store the plurality of datasets of the sensor data includes an instruction to store each dataset in a different memory buffer.

In certain embodiments, the memory including processor executable instructions to store the plurality of datasets of the sensor data includes an instruction to store each dataset in separate memory buffers within sensor electronics disposed with an on body device.

In certain embodiments, the memory including processor executable instructions further includes an instruction to transfer the plurality of datasets of the sensor data from the separate memory buffers to a receiver for signal processing.

In certain embodiments, the memory including processor executable instructions to store the plurality of datasets of the sensor data includes an instruction to store the plurality of datasets of the sensor data in separate memory partitions of a single memory device.

In certain embodiments, the memory including processor executable instructions to determine the estimated analyte level includes an instruction to determine a discrete time approximation of a filter model represented by a state space realization.

In certain embodiments, the memory including processor executable instructions to determine the estimated analyte level includes instructions to: generate a composite dataset based on a selection of all or a subset of data points from the plurality of datasets of sensor data, determine a set of filter parameters based on rates of the composite dataset, and determine the estimated analyte level based on one or more filters using the set of filter parameters and the composite dataset.

An on body device for an analyte monitoring system in certain embodiment includes an analyte sensor configured to be positioned at least partially subcutaneously, sensor electronics coupleable to the analyte sensor, wherein the sensor electronics includes a first memory buffer configured to store sensor data at a first data rate, and a second memory buffer configured to store sensor data at a second data rate.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

1. An apparatus comprising:
one or more processors configured to receive signals generated by an analyte sensor; and
a memory storing instructions which, when executed by the one or more processors, cause the one or more processors to:
store a first dataset corresponding to the signals generated by the analyte sensor at a first rate in a first memory buffer; and
store a second dataset corresponding to the signals generated by the analyte sensor at a second rate in a second memory buffer, wherein the second rate is a dynamic value based on a rate-of-change corresponding to the signals generated by the analyte sensor.

2. The apparatus of claim 1, wherein the instructions which, when executed by the one or more processors, further cause the one or more processors to:
determine that the rate-of-change corresponding to the signals has increased; and
increase the second rate at which the second dataset is stored in the second memory buffer.

3. The apparatus of claim 1, wherein the instructions which, when executed by the one or more processors, further cause the one or more processors to:
determine that the rate-of-change corresponding to the signals has decreased; and
decrease the second rate at which the second dataset is stored in the second memory buffer.

4. The apparatus of claim 1, wherein a size of the first memory buffer is different than a size of the second memory buffer.

5. The apparatus of claim 1, wherein the first memory buffer and the second memory buffer are separate memory buffers within a single memory device.

6. The apparatus of claim 1, wherein the first dataset and the second dataset have different data densities from each other.

7. The apparatus of claim 1, wherein the instructions which, when executed by the one or more processors, further cause the one or more processors to determine an estimated analyte level by:
generating a composite dataset based on a selection of all or a subset of data points from the first dataset and the second dataset;
determining a set of filter parameters based on rates of the composite dataset; and
determining the estimated analyte level based on one or more filters using the set of filter parameters and the composite dataset.

8. The apparatus of claim 1, wherein the first rate corresponds to a size of the first memory buffer, and wherein the second rate corresponds to a size of the second memory buffer.

9. The apparatus of claim 1, wherein the analyte sensor samples data at a constant sampling rate, and wherein the first rate and the second rate are each based on fractions of the sampling rate.

10. A method comprising:
receiving signals generated by an analyte sensor;
store a first dataset corresponding to the signals generated by the analyte sensor at a first rate in a first memory buffer; and
store a second dataset corresponding to the signals generated by the analyte sensor at a second rate in a second memory buffer, wherein the second rate is a dynamic value based on a rate-of-change corresponding to the signals generated by the analyte sensor.

11. The method of claim 10, further comprising:
determining that the rate-of-change corresponding to the signals has increased; and
increasing the second rate at which the second dataset is stored in the second memory buffer.

12. The method of claim 10, further comprising:
determining that the rate-of-change corresponding to the signals has decreased; and
decreasing the second rate at which the second dataset is stored in the second memory buffer.

13. The method of claim 10, wherein a size of the first memory buffer is different than a size of the second memory buffer.

14. The method of claim 10, wherein the first memory buffer and the second memory buffer are separate memory buffers within a single memory device.

15. The method of claim 10, wherein the first dataset and the second dataset have different data densities from each other.

16. The method of claim 10, further comprising determining an estimated analyte level by:
generating a composite dataset based on a selection of all or a subset of data points from the first dataset and the second dataset;
determining a set of filter parameters based on rates of the composite dataset; and
determining the estimated analyte level based on one or more filters using the set of filter parameters and the composite dataset.

17. The method of claim 10, wherein the first rate corresponds to a size of the first memory buffer, and wherein the second rate corresponds to a size of the second memory buffer.

18. The method of claim 10, wherein the analyte sensor samples data at a constant sampling rate, and wherein the first rate and the second rate are each based on fractions of the sampling rate.

19. The method of claim 10, further comprising transferring the first dataset and the second dataset to a receiver for processing.

20. An apparatus comprising:
one or more processors configured to receive signals generated by a glucose sensor; and
a memory storing instructions which, when executed by the one or more processors, cause the one or more processors to:
store a first dataset corresponding to the signals generated by the glucose sensor at a first rate in a first memory buffer; and
store a second dataset corresponding to the signals generated by the glucose sensor at a second rate in a second memory buffer, wherein the second rate is a dynamic value based on a rate-of-change corresponding to the signals generated by the glucose sensor.
